(19) United States
(12) Patent Application Publication
(10) Pub. No.: US 2006/0201507 A1
(43) Pub. Date: Sep. 14, 2006

(54) Stand-alone circle circuit with co2 absorption and sensitive spirometry for measurement of pulmonary uptake

(75) Inventor: Peter H. Breen, Irvine, CA (US)

Correspondence Address: STOUT, UXA, BUYAN & MULLINS LLP, 4 VENTURE, SUITE 300, IRVINE, CA, 92618, US

(73) Assignee: The Regents of the University of California, Oakland, CA (US)

(21) Appl. No.: 10/530,603

(22) PCT Filed: Oct. 14, 2003
(86) PCT No.: PCT/US03/32654

§ 371 Date: Mar. 16, 2006

Related U.S. Application Data
(60) Provisional application No. 60/418,197, filed on Oct. 11, 2002.

(51) Int. Cl. A61M 16/00; A62B 7/00

(52) U.S. Cl. 128/204.22; 128/204.18; 128/204.21; 128/204.23

(57) ABSTRACT

A system and method for determining the volume of oxygen taken up by the lungs of a human or veterinary patient. A closed ventilation circuit is connected to a spirometric device, which contains a quantity of oxygen. As oxygen is taken up by the patient, an equivalent volume of oxygen passes from the spirometric device into the ventilation circuit. The volume of oxygen that moves from the spirometric device may thus be measured as an indication of the volume of oxygen that has been taken up by the patient. In some embodiments, a source of make-up oxygen is connected to the ventilation circuit and the flow of make-up oxygen is adjusted as necessary to maintain a substantially constant volume of oxygen in the spirometric device. The volume of make-up oxygen is measured and serves as an indication of the amount of oxygen taken up by the patient. A valve may be used to close the spirometric device off from the ventilation circuit during all but a part of the ventilation cycle (e.g., all but the late expiratory phase) to prevent substantial pressure or movement excursions within the spirometric device

### RELATED APPLICATION

This application claims priority to U.S. Provisional Patent Application No. 60/418,197 entitled “Stand-Alone Closed Ventilating Circle With CO Absorption and Sensitive Spirometry for Measurement of Pulmonary Uptake During Anesthesia” filed on Oct. 11, 2002, the entirety of which is expressly incorporated herein by reference.

### STATEMENT REGARDING FEDERALLY SPONSORED RESEARCH OR DEVELOPMENT

Work connected with this invention was supported in part by the National Institutes of Health Grant # 42637. The United States Government may have rights in this invention.

### FIELD OF THE INVENTION

The present invention relates generally to biomedical devices and methods and more particularly to devices and methods for measuring lung function in human or veterinary patients during anesthesia or mechanical ventilation, or assessment of pulmonary or metabolic function.

### BACKGROUND OF THE INVENTION

Oxygen Uptake as a Measurement of Lung Function

Normally, humans respire at a rate of around twelve (12) breaths per minute. The average human inhales about 0.5 liters of air per breath or about 6 liters of air per minute. The normal oxygen uptake (\dot{V}ₒ_{₂}) in adult humans is about 300 ml/minute, which represents an uptake of only about 25% of the oxygen available in inhaled room air. Oxygen uptake in the lungs can decrease when: a) the partial pressure of oxygen in the inhaled air is substantially reduced, b) blood flow through the lungs is lessened (e.g., due to reduced cardiac output, the presence of pulmonary emboli or other disruptions of pulmonary circulation), c) diffusion across the alveolar membranes is impaired (i.e., due to pulmonary edema, atelectasis, granulomatous disease, chemical inhalation bum, etc.), d) the normal expansion and/or collapse of the lung is impaired (e.g., due to pneumothorax, emphysema, etc.), e) the oxygen carrying capacity of the blood is impaired (e.g., due to a fall in the number of oxygen carrying red blood cells, hemorrhage, anemia, etc.), f) the airway(s) become obstructed (e.g., due to mucous plugging, chronic obstructive pulmonary disease, etc.), g) the body tissue's consumption of oxygen decreases (e.g., anaerobic metabolism, hypothermia, cyanide poisoning, etc.) and other causes.

Thus, a measurement of oxygen uptake (\dot{V}ₒ_{₂}) can be valuable in monitoring patients during surgery or in critical care or emergency settings, especially those wherein the patient is undergoing mechanical ventilation.

Traditional Spirometry

Devices known as “spirometers” have been used for many years to measure static and dynamic lung volumes. There are numerous types of spirometers available today. In general terms, displacement spirometers are those in which the patient breathes into a closed space causing an indicator to move up and down, thereby indicating the volume of breath inhaled and exhaled by the patient. There are two basic types of displacement spirometers, “dry seal” and “water seal.” Each type has certain advantages and disadvantages. In water-seal displacement spirometers, a concave “bell” (e.g., a cylinder or drum) floats on water and traps a certain volume of gas within the bell. The patient breathes through a tube that extends into the interior of the bell where the gas is trapped. The resultant changes in gas volume within the bell cause the bell to move up and down in the water. The distance by which the bell moves up and down is a measure of the volume of gas inhaled and exhaled during each breath. Water seal spirometers typically provide accurate data but also require substantial care and maintenance. In some designs, the use of water can cause corrosion of parts and requires substantial ongoing maintenance.

Dry seal displacement spirometers typically use a rubber or plastic bellows that expands and contracts as the patient breathes. Due to inherent resistance in the bellows, dry seal displacement spirometers tend to be somewhat less accurate than those that are water-sealed. They can, however, require less maintenance due to the fact that they are not required to contain water.

Spirometers have heretofore been used to directly measure static and dynamic lung volumes, including tidal volume (\dot{V}_{T}), vital capacity (VC) and forced expiratory volume (FEV). In order for measurements to be accurate, corrections should be made for differing temperature and humidity of the gas in the lungs. Special look-up tables are available to facilitate the making of such corrections. Spirometers are also used for tests involving continuous breathing such as residual volume (RV). Spirometers have also been used in the determination cardiac output (liters of blood/minute) using a technique known as the Fick method. Determination of cardiac output by the Fick method requires a calculation of oxygen consumption. This is achieved using a spirometer in conjunction with a carbon dioxide absorber. The oxygen consumption is the reduction in volume/minute of the quantity of gas in the spirometer.

Although spirometers have been used for direct measurement of lung volumes and indirect measurement of cardiac output, spirometers have not previously been adapted for determination of oxygen uptake (\dot{V}ₒ_{₂}). Instead, comparatively expensive calorimetric equipment is typically required for monitoring of oxygen uptake (\dot{V}ₒ_{₂}).

Given the emerging importance of oxygen uptake (\dot{V}ₒ_{₂}) monitoring in anesthesia and critical care, there exists a need in the art for the development of a relatively inexpensive, simple system for measurement of oxygen uptake (\dot{V}ₒ_{₂}) without the need for complex analytical instrumentation.

### SUMMARY OF THE INVENTION

The present invention provides a system and method for accurate, non-invasive, measurement of O₂ uptake (\dot{V}ₒ_{₂}) in human or veterinary patients. The system generally comprises a spirometric device (e.g., a wet or dry seal displacement-type spirometer) filled with oxygen (pure oxygen or gas containing some known % of oxygen) connected to the expiratory flow conduit of a ventilation circuit (e.g., a circle circuit). A valve may be positioned between the expiratory flow conduit and the oxygen-containing interior of the spirometric device. Such valve may be opened only during late expiration to prevent backflow into the spirometer. In embodiments where no new oxygen is added to the ventilation circuit, the volume of oxygen within the spirometer will change in direct relationship to the oxygen volume uptake occurring in the expiratory limb of the ventilation circuit when the valve is open. Thus, the amount of downward movement of the spirometer (e.g., the wet or dry sealed bell, bellows or other variable volume container) is indicative of the amount of oxygen that has been taken up by the patient's lungs (\dot{V}ₒ_{₂}). Alternatively, in embodiments where a source of make-up oxygen is connected to the ventilation circuit, the inflow of make-up oxygen into the ventilation circuit may be adjusted, as required, to cause the position of the volume of oxygen within the spirometric device to remain substantially unchanged from breath to breath. In such embodiments, the amount (flow) of make-up oxygen required to cause the volume of oxygen within the spirometric device to remain substantially constant will be equal to the amount of oxygen being taken up by the patient's lungs (\dot{V}ₒ_{₂}).

Further aspects and advantages of this invention will become apparent to those of skill in the art upon reading and understanding of the following detailed description and the drawings to which it refers.

### BRIEF DESCRIPTION OF THE DRAWINGS

FIG. 1 is a diagram of a typical displacement type, water-sealed spirometer of the prior art.

FIG. 2 is a diagram of one embodiment of the system of the present invention useable for measuring oxygen uptake in human or veterinary patients.

### DETAILED DESCRIPTION OF THE INVENTION

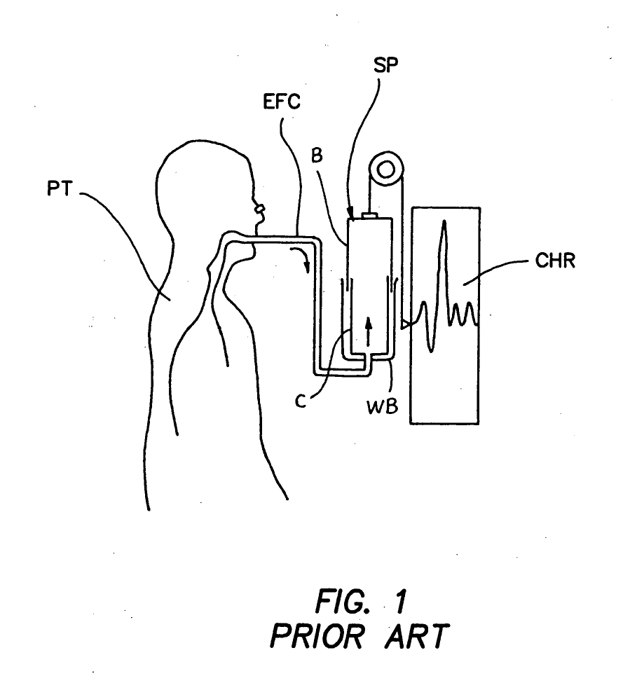

FIG. 1 shows a typical water-seal, displacement spirometer of the prior art. As shown, the spirometer comprises a water bath WB having a cylinder that comprises a lower portion LP and a bell B positioned therein such that the bell B will float up and down in accordance with the volume of gas contained within the cylinder. The bottom rim of the bell B is submerged within water contained in the water bath WB such that the water forms a seal and prevents gas from escaping the cylinder. A chart recorder CHR records the up/down movement of the bell B. The patient PT breaths through conduit C causing the bell to move up each time the patient exhales and down each time the patient inhales. From the chart recording, one may determine lung volume measurements such as tidal volume (\dot{V}_{T}), vital capacity (VC) and forced expiratory volume (FEV).

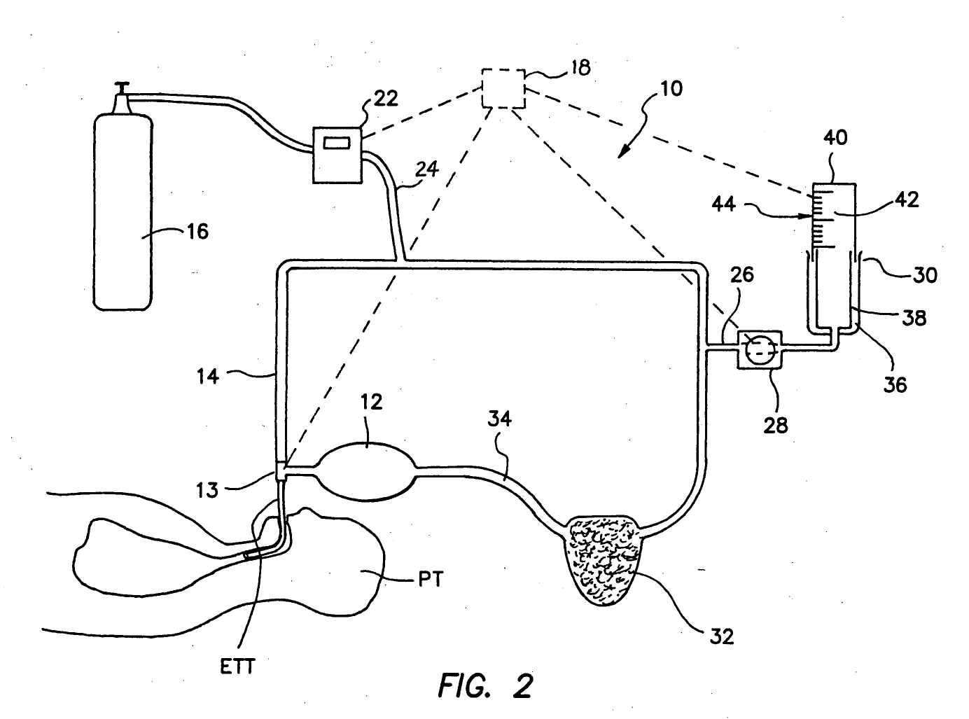

FIG. 2 shows a system for measurement of oxygen uptake (\dot{V}ₒ_{₂}), in accordance with the present invention. As shown, the system 10 comprises a ventilation device such as a bag 12 (e.g., a self inflating ‘Ambu’ bag available commercially from a variety of manufacturers) connected to an endotrachaeal tube ETT (or other airway apparatus such as a mask, laryngeal mask airway, nasotrachaeal tube, tracheostomy tube, etc.) via a three-way valve 13. The three-way valve is typically incorporated into the ventilation bag 12. An expiratory flow conduit 14 is also connected to the three-way valve 13. When the bag 12 is compressed inspiratory gas flows through the three-way valve 13, through the endotrachaeal tube ETT and into the patient's lungs. When the patient exhales, the three-way valve 13 changes position and the exhaled respiratory gas flows into the expiratory flow conduit 14. The expiratory flow conduit is connected to a carbon dioxide absorber 32 (e.g., SODASORB® 4-8 IND N MED, Daerx® Container Products, Cambridge, Mass. or ThermHOAbsorb™, Raincoat Industries, Inc., Louisville, Ky.) such that expired gas flowing through the expiratory flow conduit 14 will flow though the carbon dioxide absorber 32 and all of the carbon dioxide contained in the expired gas will be removed. The gas exiting the carbon dioxide absorber 32 then flows into the inspiratory flow conduit 34, through the bag 12 and once again into the patient's lungs. A water-seal spirometer 44 (e.g., a custom made water seal spirometer having a 4 cm ID) is connected to the expiratory flow conduit 14 by side tube 26. The spirometer 44 comprises a water bath 30 filled with water 36 and a telescoping oxygen-containing cylinder formed of an upper bell member 42 and a lower member 38. The bottom edge or rim of the bell member 42 is submerged within the water 36 such that a liquid seal is formed and oxygen is prevented from escaping from the interior of the cylinder. A linear scale 40 (e.g., calibrated in millimeters) is printed in the outside of the bell member 42, as shown. A quantity of oxygen (e.g., preferably pure oxygen) is contained within the cylinder, beneath the bell member 42.

A valve 28 is positioned on side tube 26. This valve 28 is opened only during late expiration to prevent back-flow into the spirometer 44. When the valve 28 is open, oxygen will flow out of the spirometer 44, through tube 26 and into the expiratory flow tube 14 to make up for oxygen that has been taken up by the patient's lungs, provided that no new or make-up oxygen has been added to the circuit. When no make-up oxygen is added to the circuit, the volume of oxygen that moves out of the spirometer 44 will be equal to the volume of oxygen that has been taken up by the patient's lungs. As oxygen passes out of the spirometer, the bell 42 will move downwardly and the amount of downward movement of the bell 42 may be read on the linear scale 40 and correlated directly to oxygen uptake (\dot{V}ₒ_{₂}).

Optionally, a source of oxygen 16 may be connected to the ventilation circuit by make-up oxygen supply tube 24. A flowmeter 22 may be positioned on make-up oxygen supply tube 24 to vary the flow of make-up oxygen into the ventilation circuit. The flowmeter 22 may be adjusted as needed until substantially no movement of the bell 42 is observed from breath to breath. (e.g., less than 1 mm movement after each breath), at which time the volume of oxygen flowing through the make-up oxygen line 24 will be substantially the same as the volume of oxygen being taken up by the patient's lungs.

Optionally, a controller 18 (e.g., a microprocessor, computer or other programmable control device) may be connected to the bag 12 or other ventilation device or to one or more sensors (e.g., pressure and/or flow sensor(s)) to monitor the changes in phase of the ventilation cycle. Such controller 18 may send control signals to valve 28 to cause valve 28 to open and close at desired times during the ventilation cycle (e.g., open only during the late expiratory phase and closed during the rest of the cycle). In embodiments where make-up oxygen is infused into the ventilation circuit through oxygen line 24, the controller 18 may also be connected to the spirometer 44 and may send control signals to the flowmeter 22 to adjust the flow of make-up oxygen as necessary to prevent substantial changes in the volume of oxygen contained in the spirometer 44. Any suitable monitoring apparatus may also be connected to the system to compute the movement of the spirometer 44 and/or amount of make-up oxygen added and to provide a display of the oxygen uptake (\dot{V}ₒ_{₂}) determined by the system 10. An important design element is that the self-inflating ventilating bag (or any other implementation of manual or automatic ventilation such as a mechanical ventilator, bellows, etc.) return to exactly the same pre-inspiration volume before each inspiration. This element of the invention ensures that the change in position of the spirometer accurately measures the pulmonary oxygen uptake (\dot{V}ₒ_{₂}) of the patient during steady state conditions.

The stand-alone circuit facilitates normal mechanical ventilation, absence of physiological gas leaks, complete CO₂ absorption, and the ability to measure small changes (1 ml) in circuit end-expired volume via the valve and precision spirometer. The use of this system as a reference standard measurement of \dot{V}ₒ_{₂} will provide for calibration and development of practical \dot{V}ₒ_{₂} systems during anesthesia, particularly Vₒ_{₂} per breath. \dot{V}ₒ_{₂} will become an essential monitor to detect non-steady state critical events and changes in tissue metabolism during anesthesia. Additionally, this system may be used as a relatively simple apparatus for measuring oxygen uptake \dot{V}ₒ_{₂} in a variety of patients.

When constructed as described using adult-sized components, the system 10 can be used to deliver a wide clinical range of ventilation (tidal volume 250-1200 ml; frequency, up to 20 br/min). Timely opening and closing of the valve 28 minimizes large oscillations in the spirometer, which may allow end-expired readings of the height of the spirometer bell 44 within approximately 1 mm on the scale 44 (i.e., 1.3 ml of volume change).

The spirometer 44 also functions as an O₂ reservoir. This measurement of \dot{V}ₒ_{₂} provides a reference standard needed for the development of practical \dot{V}ₒ_{₂} measurements during anesthesia or critical care ventilation. The use of this system may facilitate the measurement and use of oxygen uptake per breath (Vₒ_{₂}_{,br}) as a monitored variable during anesthesia to detect non-steady state critical events and changes in tissue metabolism.

Although the invention has been described herein with reference to certain specific embodiments or examples, it will be appreciated that various changes, additions or alterations may be made to the specifically described embodiments and examples without departing from the intended spirit and scope of the invention. Thus, it is intended that all such additions, deletions and alterations be included within the scope of the following claims

1. A system for measuring oxygen uptake in a human or veterinary patient, said system comprising:
an inspiratory flow conduit for delivering a flow of inspiratory gas to the lungs of the patient;
an expiratory flow conduit for carrying expired gas from the lungs of the patient;
a ventilation apparatus attached to the inspiratory flow conduit for moving inspiratory gas through the inspiratory flow conduit toward the lungs of the patient; and, a spirometric device comprising a chamber, which contains a volume of oxygen and an indicator for indicating changes in the volume of oxygen contained within the chamber;
the spirometric device being connected to the ventilation circuit such that the volume of oxygen contained in the chamber will vary relative to the volume of oxygen taken up by the patient.

2. A system according to claim 1 further comprising a carbon dioxide absorber connected to the system such that gas from the expiratory flow conduit will pass through the carbon dioxide absorber where carbon dioxide will be removed from the gas and the gas will subsequently flow from the carbon dioxide absorber into the inspiratory flow conduit

3. A system according to claim 1 further comprising a valve positioned between the spirometry device and the expiratory flow conduit, said valve being open only during a late portion of the expiratory phase of the ventilation cycle, thereby preventing substantial pressure variations within the spirometric device as a result of inhalation and exhalation.

4. A system according to claim 3 wherein the valve is adapted to be opened and closed in response to control signals and wherein the system further comprises a control device which sends control signals to the valve to cause the valve to open and close at predetermined points on the ventilation cycle.

5. A system according to claim 4 wherein the controller is operative to cause the valve to open at approximately the end of each expiration and to close at approximately the beginning of each inspiration.

6. A system according to claim 1 further comprising a source of make-up oxygen connected to the ventilation circuit.

7. A system according to claim 6 further comprising a flow control apparatus for controlling the flow of make-up oxygen into the ventilation circuit.

8. A system according to claim 7 wherein the flow control apparatus is adapted to increase or decrease the flow of make-up oxygen into the ventilation circuit in response to control signals and wherein the system further comprises a control device which sends control signals to the flow control apparatus to increase or decrease the flow rate of make-up oxygen into the ventilation circuit.

9. A system according to claim 8 wherein the controller is operative to cause the flow control apparatus to increase or decrease the flow of make-up oxygen as required to prevent more than a predetermined amount of change in the volume of oxygen contained in the cylinder.

10. A system according to claim 1 wherein the spirometric device comprises a water sealed spirometer.

11. A system according to claim 1 wherein the spirometric device comprises a dry sealed spirometer.

12. A system according to claim 1 wherein the chamber moves in relation to the volume of oxygen contained within the chamber and wherein the indicator comprises an indicator of chamber movement.

13. A system according to claim 12 wherein the indicator comprises a scale marked on the chamber to indicate the distance by which the chamber has moved.

14. A system according to claim 1 wherein the ventilation apparatus comprises a bag, ventilator, bellows or other manual or automatic ventilating apparatus.

15. A system according to claim 1 wherein the ventilating apparatus returns to the same volume prior to each breath.

16. A method for determining oxygen uptake in a human or veterinary patient, said method comprising the steps of:
A. providing a closed ventilation circuit that comprises i) an expiratory flow conduit for carrying expired gas from the lungs of the patient, ii) a ventilation apparatus attached to the inspiratory flow conduit for moving inspiratory gas through the inspiratory flow conduit toward the lungs of the patient and iii) a spirometric device comprising a chamber which contains a volume of oxygen and an indicator for indicating changes in the volume of oxygen contained within the chamber, wherein the spirometric device is connected to the expiratory flow conduit such that the volume of oxygen contained in the chamber of the spirometric device will vary relative to the volume of oxygen taken up by the patient;
B. connecting the ventilation circuit to the patient such that the patient will inhale and exhale through the ventilation circuit and
C. determining the change in the volume of oxygen contained in the chamber of the spirometric device as an indication of oxygen uptake by the patent.

17. A method according to claim 16 wherein the ventilation circuit provided in Step A further comprises a carbon dioxide absorber for absorbing at least some of the carbon dioxide contained in respiratory gas expired by the patient.

18. A method according to claim 16 wherein the spirometric device provided in Step A comprises a water seal spirometer.

19. A method according to claim 16 wherein the spirometric device provided in Step A comprises a water seal spirometer.

20. A method according to claim 16 wherein the ventilation circuit is connected to a source of make-up oxygen from which oxygen is infused into the ventilation circuit and wherein the Step C comprises:
adjusting the amount of oxygen that is infused into the ventilation circuit such that the volume of oxygen in the chamber of the spirometric device does not change substantially; and
determining the volume of oxygen that has been added to the ventilation circuit as an indication of the amount of oxygen that has been taken up by the patient.

21. A method according to claim 16 wherein the ventilation circuit provided in Step A further comprises a valve positioned between the spirometric device and the ventilation circuit such that when the valve is open the chamber of the spirometric device is in fluidic communication with the ventilation circuit and when the valve is closed the spirometric device is not in fluidic communication with the ventilation circuit, and wherein the method further comprises the step of:
opening and closing the valve at differing times in the ventilation cycle to prevent substantial pressure or movement variations within the chamber of the spirometric device.

22. A method according to claim 21 wherein the valve is open only during the late expiratory portion of the ventilation cycle
